(19) United States
(12) Patent Application Publication
(10) Pub. No.: US 2009/0240257 A1
(43) Pub. Date: Sep. 24, 2009

(54) Limit Control for IOL Injector

(76) Inventor: Rolf Meyer, Biel-Bienne (CH)

Correspondence Address: LAW OFFICES OF JERRY A. SCHULMAN, 1S376 SUMMIT AVENUE, COURT C, OAKBROOK TERRACE, IL, 60181, US

(21) Appl. No.: 11/744,884

(22) Filed: May 7, 2007

(51) Int. Cl. A61F 9/007

(52) U.S. Cl. 606/107

(57) ABSTRACT

An injector having a hollow cylindrical body having a first, discharge end and a second, plunger end, and a plunger which is axially slidable within the body. A first end of the plunger is adapted to contact either an IOL or a cartridge containing an IOL and to force the IOL out of the injector or cartridge into the eye. A second end of the plunger has a knob or stop proximate the pushing end, used to grip the plunger and move it into the housing. Located on the housing proximate the second end is a micrometer barrel rotatably and threadedly mounted to the injector housing. When the plunger is fully pushed into the injector housing, the knob contacts the micrometer collar thus halting the inward movement of the plunger. The micrometer collar is rotatably adjustable with respect to the housing thereby altering the position at which the plunger knob contacts the micrometer dial and, thereby, adjusting the distance to which the the plunger extends toward or past the discharge end of the injector.

This invention relates to injectors for foldable intraocular lenses and, more particularly, to a control for adjusting the length to which the injector plunger is extended.

### BACKGROUND OF THE INVENTION

A common ophthalmological surgical technique for treating cataracts and other diseases of the lens is to fractionate, emulsify and remove the diseased lens and replace it with a synthetic, plastic lens. To do so, an incision is made in the eye through which the diseased lens is removed and through which the new lens is inserted.

It is desirable to make the incision for removing the diseased lens and inserting the new lens to be as small as possible. This aids in shortening recovery time and limits the leakage of fluid through the incision after surgery without requiring sutures.

When originally introduced, replacement intraocular lenses (IOLs) were made from rigid plastics such as polymethylmethacrylate and required a relatively large corneal incision through which the lens would be inserted. Such incisions had to be sutured to limit the loss of fluid from the eye. The development of the soft lens, made from materials such as hydrogels, silicones and soft acrylics made it possible to fold or roll the lens prior to insertion. It is now a common surgical technique to fold such an IOL, insert it through the incision and allow the lens to unfold and position itself within the eye. When folded, the lens needs a much smaller incision for insertion than if the lens were inserted in its fully unfolded state.

Examples of foldable IOLs and systems for injecting them are well represented in the prior art.

U.S. Pat. No. 5,947,976 (Van Noy et al.) teaches and describes an asymmetric IOL injection cartridge having an asymmetric bore. The IOL is inserted into the cartridge in its unfolded state, and when pushed through the cartridge, is partially folded when it exits the cartridge tip.

U.S. Pat. No. 5,976,150 (Copeland) teaches and describes an IOL injection system using a foldable substrate to compress and fold an IOL around an outer edge of the substrate. The folded assembly is then placed in an injector and expressed through the injector nozzle into the eye.

U.S. Pat. No. 6,083,231 (Van Noy et al) teaches and describes an asymmetric IOL injection cartridge. This patent is a continuation in part of the previously described U.S. Pat. No. 5,947,976 and adds to the disclosure in the '976 patent a peg used to engage the haptic on an IOL to keep the haptic in position as the lens is injected through the injector nozzle.

U.S. Pat. No. 6,143,001 (Brown et al) teaches and describes an asymmetric intraocular lens injection cartridge. This patent is a continuation in part of the previously described U.S. Pat. No. 6,083,231 as well as the '976 patent. Brown et al adds to the teachings of the prior mentioned patents a modified asymmetric bore which acts to fold the IOL as it is forced through the bore and the cartridge nozzle.

U.S. Pat. No. 6,398,789 (Capetan) teaches and describes an IOL injector cartridge similar in construction to the cartridge shown in the '976, '231 and '001 patents and which adds to the elements of those patents a heat-retention agent to keep the IOL warmed during the injection process.

U.S. Pat. No. 6,537,283 (Van Noy) teaches and describes an IOL shipping case and injection cartridge comprising an injection cartridge to which a case for an unfolded IOL is hingedly attached. The case can be rotated to align the lens with the central bore of the cartridge to let the lens thereafter be pushed through the cartridge and expressed through the cartridge tip.

U.S. Pat. No. 4,681,102 (Bartell) teaches and describes apparatus and method for insertion of an IOL where the system consists of an injector and a foldable load chamber within which the unfolded lens is placed. The chamber is then folded shut thereby folding the lens in half and a plunger is used to force the lens from the load chamber through a tip and into the eye.

U.S. Patent Application Publication No. 2004/0117012 (Vincent) teaches and describes an injector for an intraocular lens, the injector having a hollow cylindrical body and a plunger that, when pushed into the body, ejects a folded IOL from the injector nozzle.

U.S. Pat. No. 6,607,537 (Binder) teaches and describes an injector for implanting a folded intraocular lens, container for storing and transporting the injector and method for injecting the lens in a folded state. The injector has a hollow body within which a folded IOL is placed and a plunger that, when pressed into the body, forces the IOL outward through the injector nozzle.

The injectors typified by the foregoing references have plungers with a maximum travel length but lack an adjustment to select a travel length less than maximum. For the surgeon, selecting a travel length is necessary to accommodate variations in the physiology of individual eyes. Some patients have deeper-set eyes or eye lengths that are longer or shorter than others. Using commonly available injectors requires the surgeon to observe the progress of the IOL into the eye and stop pushing the plunger when the surgeon feels the IOL is correctly positioned.

A prominent feature of the present invention is reproducibly preset mechanism to intercept the plunger at a point short of its maximum travel and thus allow the surgeon to preselect a length of travel prior to the injection of the IOL.

### SUMMARY OF THE INVENTION

The present invention comprises an injector having a hollow cylindrical body having a first, discharge end and a second, plunger end, and a plunger which is axially slidable within the body. A first end of the plunger is adapted to contact either an IOL or a cartridge containing an IOL and to force the IOL out of the injector or cartridge into the eye. A second end of the plunger has a knob or stop proximate the pushing end, used to grip the plunger and move it into the housing. Located on the housing proximate the second end is a micrometer barrel rotatably and threadedly mounted to the injector housing. When the plunger is fully pushed into the injector housing, the knob contacts the micrometer collar thus halting the inward movement of the plunger. The micrometer collar is rotatably adjustable with respect to the housing thereby altering the position at which the plunger knob contacts the micrometer dial and, thereby, adjusting the distance to which the the plunger extends toward or past the discharge end of the injector.

These and other aspects of the present invention will become apparent upon consideration of the accompanying description and the accompanying drawings in which:

### BRIEF DESCRIPTION OF THE DRAWINGS

FIG. 1 is a perspective view of a prior art cartridge injector;

FIG. 2 is a perspective view of a preferred embodiment of the present invention showing the plunger fully withdrawn from the housing;

FIG. 3 is a perspective view of the injector of FIG. 2 showing the plunger fully pushed into the injector housing;

FIG. 4 is a lateral view of the injector shown in FIG. 2;

FIG. 5 is a lateral view of the injector shown in FIG. 3;

FIG. 6 is a perspective view of a second embodiment of the present invention showing an injector without an external damping spring;

FIG. 7 is a lateral view of the injector shown in FIG. 6 with the plunger fully withdrawn from the housing;

FIG. 8 is a lateral view of the injector shown in FIG. 6 with the plunger fully pushed into the housing;

FIG. 9 is a detailed view of the micrometer dial;

FIG. 10 is a lateral view of an adjustment mechanism with preset discrete adjustment steps or positions; and

FIG. 11 is an enlarged detail of the adjustment mechanism of FIG. 10.

### DETAILED DESCRIPTION OF THE DRAWINGS

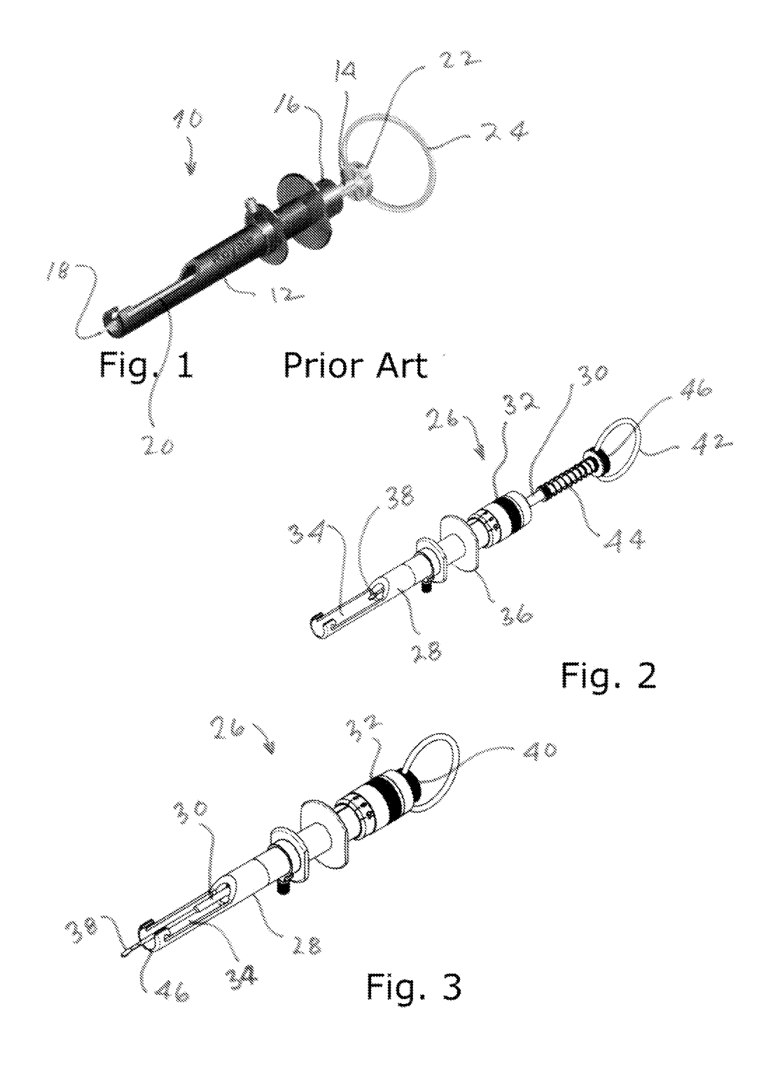

Referring now to FIG. 1, numeral 10 identifies a prior art IOL injector sold by Asico LLC of Westmont, Ill. as its Model No. AE-9045. Injector 10 has an injector body 12 which is hollow and generally cylindrical in cross section. A plunger 14 is slidably received at first injector body end 16. Plunger 14 terminates in a pusher end 18 adapted to contact and push an IOL.

Injector 10 is intended for use with a cartridge into which the IOL is placed and the cartridge is held in cartridge slot 20. When plunger 14 is pushed into housing 12, pusher end 18 enters the cartridge, contacts the IOL and forces it out through the cartridge nozzle into the eye. Other types of injectors have hollow cylindrical bodies terminating in a nozzle and have the folded IOL placed within the injector body itself, whereupon the pusher end of the plunger contacts the IOL and forces it through the injector nozzle. The present invention finds utility on injectors designed to handle only a specific type of cartridge or many types of cartridges, or injectors that inject an IOL directly with no cartridge.

At the actuation end of the injector 10, plunger 14 terminates in a knob 22 to which a thumb ring 24 is attached. Knob 22 and ring 24 give the surgeon a firm and positive grip when using injector 10.

When plunger 14 is pushed into housing 12 the IOL is forced either from the cartridge or from the injector body. When using such an injector, the surgeon must determine either by feel or by eye, how far plunger 14 must be advanced into body 12 to force the IOL into a proper position within the eye.

Referring now to FIG. 2, the numeral 26 identifies an injector having a hollow cylindrical body 28 which slidably receives a plunger 30 at a micrometer plug 32. The injector shown in FIG. 2 has a cartridge receiving slot 34 and a finger grip 36. Plunger 30 terminates at one end with a pusher 38 movable axially and slidably within housing 28 and, at the other end, a plunger knob 40 and a thumb ring 42. In the embodiment shown in FIG. 2, a damping spring 44 is mounted concentrically about plunger 30 intermediate knob 40 and micrometer plug 32. Spring 44 provides a positive “feel” to the surgeon when, as plunger 30 is advanced into housing 28, spring 44 is compressed between micrometer plug 32 and knob 40. When plunger 30 has been advanced a sufficient distance to eject the IOL from either the cartridge or the injector nozzle, spring 44 aids in withdrawing pusher 38 by expanding against knob 40 and moving plunger 30 rearward. In FIG. 2, plunger 30 is shown in a position prior to the insertion of an IOL.

Referring now to FIG. 3, injector 26 is shown with plunger 30 fully axially advanced into housing 28. As shown in FIG. 3, pusher end 38 thus extends fully through cartridge slot 34 and extends beyond injector discharge end 46.

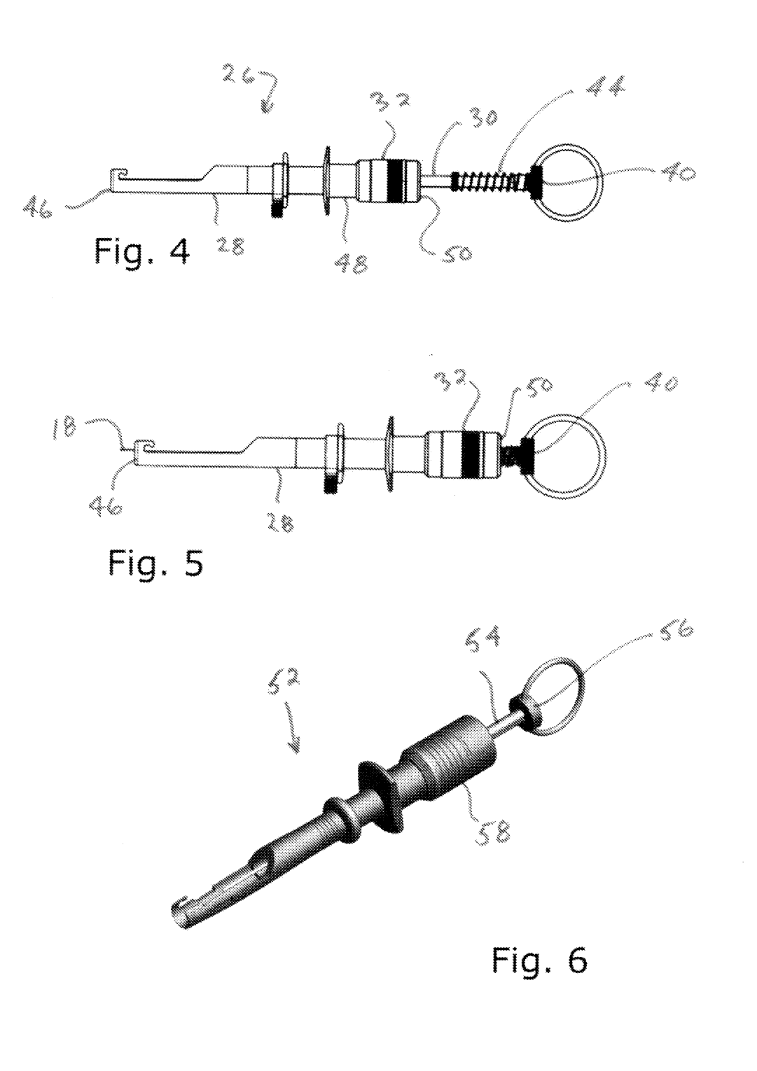

Referring now to FIG. 4, injector 26 is shown in a lateral view with plunger 30 withdrawn from housing 28. Micrometer plug 32 is threadably and rotatably attached to housing 28 such that micrometer plug 32 overlaps injector body end segment 48. When micrometer knob 32 is rotated in a first direction, it moves relative to end portion 48 in an axial direction toward discharge end 46. This allows plunger 30 to travel a greater distance before spring 44 is compressed tightly against micrometer plug 32 by knob 40, thereby stopping the advance of plunger 30 into housing 28. When rotated in the opposite direction, micrometer plug 32 moves rearwardly or away from delivery end 46, thus shortening the distance plunger 30 can travel before being stopped against end surface 50 of micrometer plug 32.

Referring now to FIG. 5, the injector of FIG. 4 is shown with plunger 30 fully pushed into housing 28 and with spring 44 compressed fully against micrometer plug surface 50 by knob 40. This is the maximum distance plunger 30 can travel, and marks the maximum distance pusher end 18 can extend toward or past discharge end 46.

Referring now to FIG. 6, the numeral 52 identifies an injector having a plunger 54 terminating in a knob 56. Plunger 54 is slidably received by a micrometer plug 58 but with no external damping spring. It is well known in the art to provide such injectors either with simply a sliding action undamped by any spring or an internally positioned spring. The remaining components of injector 52 correspond to those discussed in connection with FIGS. 2-6 above. In particular, micrometer plug 58 is threadedly mounted to injector 52 and is thus axially adjustable as described above.

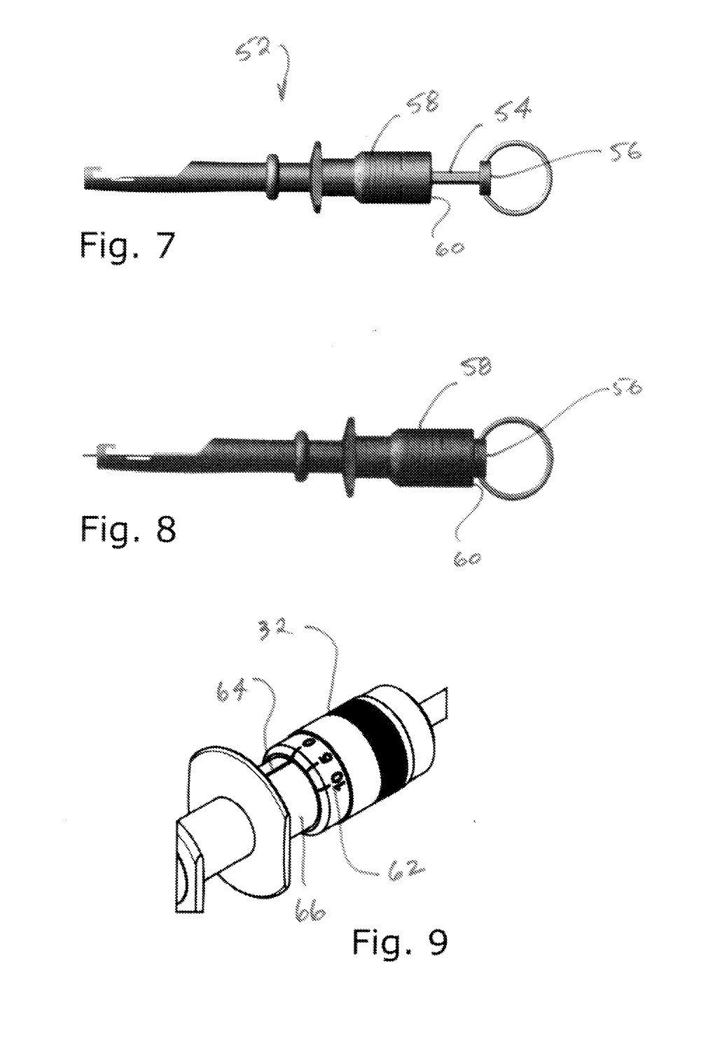

Referring now to FIG. 7, a lateral view of injector 52 is shown with plunger 54 withdrawn from injector 52.

Referring now to FIG. 8, the injector of FIG. 7 is shown with plunger 54 fully advanced into injector 52, and with knob 56 in contact with and thereby limited by rear surface 60 of micrometer plug 58. As discussed above, the position of micrometer plug 58 determines the extent of travel of plunger 54 and, thereby, the distance the pusher end of plunger 54 will travel.

Referring now to FIG. 9, an enlarged detailed view of micrometer plug 32 is shown, it being understood that the features of micrometer plug 32 are common to a micrometer plug 58 as well. Plug 32 has a numbered scale 62, divisions of which, when plug 32 is rotated, come into and out of registry with a scribe line 64 formed on rear housing 66 of injector 26. In a well-known fashion, the end position of pusher 38 can be reproduceably and accurately set by adjusting micrometer plug 32 to bring a selected scale marking 62 in registry with scribe line 64.

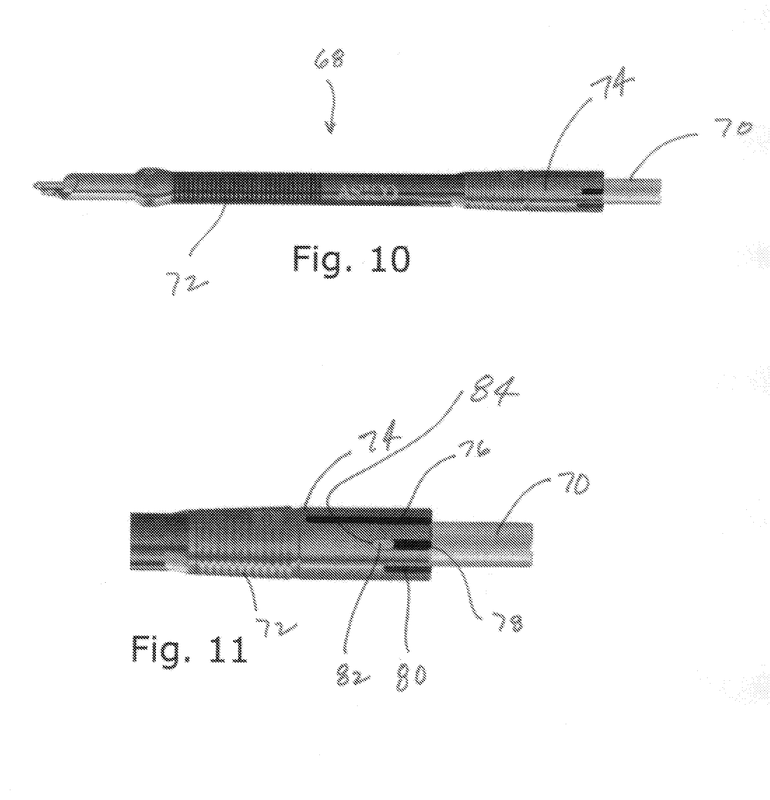

Referring now to FIG. 10, the numeral * identifies an injector having a travel adjustment which features discrete, stepped settings rather than the continuously-adjustable action of a micrometer dial.

[insert description after drawings received from ASICO]

In this fashion, a single injector can be used with a variety of cartridge configurations which may vary in the length of travel of pusher 38 required to expel an IOL from the cartridge.

The foregoing invention can readily be adapted and used with injectors having an internal spring, an external spring, a combination of both internal and external springs, or a plunger having no damping spring at all, relying on the frictional engagement between the plunger and the housing to give the injector its “feel”. The invention can also readily be adapted for use with injectors made from metal or plastic.

Referring now to FIG. 10 the numeral 68 identifies a surgical instrument having a plunger 70 received axially into body 72. An adjusting barrel 74 limits the distance plunger 70 can travel, as described below.

In FIG. 11, barrel 74 is shown in an enlarged view as having slots 76, 78 and 80 formed in a direction parallel to the longitudinal axis of instrument 68. Preferably, each said slot has a different length, corresponding to a selected distance of travel for plunger 70. The number and size of such slots is selected to create a range of movement suitable for the use of instrument 68.

A stop tab 82 is formed on or attached to plunger 70 and is sized and shaped to fit into slots 76, 78 or 80. As seen in FIG. 11, when tab 82 is aligned with and inserted into slot 78 the inward axial movement of plunger 70 will be interrupted when tab 82 reaches slot end 84.

Plunger 70 is pulled outward from body 72 and stop tab 82 is aligned with a selected slot to make possible a selected distance of travel. This mechanism can be substituted for the micrometer mechanism described heretofore to provide the adjustability required for the injection of an IOL.

While the foreging describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

I claim:

1. In an instrument adapted to insert an intraocular lens into an eye, said instrument of the type having a hollow body having first and second ends, a plunger having a first, pusher end and a second, handle end, said plunger being axially slidably received within said hollow body whereby said handle end extends from a first said body end and said handle end is movable toward said second body end thereby moving said pusher end toward said second body end, the improvement comprising:
means for stopping the motion of said plunger toward said body end,
said stopping means formed on said plunger proximate said handle end; and
means for adjustably limiting the distance said plunger may be moved,
said adjustable limiting means comprising a micrometer plug threadably attached to said body at said first body end allowing said plug to be axially adjusted with respect to said body and which, thereby, allows the position at which said stopping means contacts said plug to be adjusted, thereby changing the limit to which said pusher end may be extended toward said second instrument end.
